(19) United States
(12) Patent Application Publication
(10) Pub. No.: US 2020/0060880 A1
(43) Pub. Date: Feb. 27, 2020

(54) Methods for Protecting Skin

(71) Applicant: Adam Kolom, Encino, CA (US)

(72) Inventor: Adam Kolom, Encino, CA (US)

(21) Appl. No.: 16/112,573

(22) Filed: Aug. 24, 2018

(51) Int. Cl. A61F 13/02; A61L 26/00

(52) U.S. Cl. CPC A61F 13/0243; A61L 26/0052; A61F 2013/00387; A61L 2300/424; A61L 2300/452; A61L 26/0071

(57) ABSTRACT

A method of using a film-forming material for pre-treating an area of skin to reduce friction-related skin irritation, resulting in a reduced risk of acquiring skin chafing and/or blisters, such as may be caused by the wearing of shoes that abrade skin, and particularly including heels, said method comprising the steps of providing a dispensing device containing a film-forming material that includes a friction-reducing and/or friction-blocking element, and applying such film-forming material to an area of skin to reduce friction and thereby reduce the risk of acquiring chafing and/or a blister.

This application claims the priority of U.S. Provisional Application No. 62/549,856, filed Aug. 24, 2017.

A method of using a film-forming material for pre-treating an area of skin to reduce friction-related skin irritation, resulting in a reduced risk of acquiring skin chafing and/or blisters, such as may be caused by the wearing of shoes that abrade skin, and particularly including heels, said method comprising the steps of providing a dispensing device containing a film-forming material that includes a friction-reducing and/or friction-blocking element, and applying such film-forming material to an area of skin to reduce friction and thereby reduce the risk of acquiring chafing and/or a blister.

### FIELD OF THE INVENTION

The present invention relates to methods of skin treatment and in particular to skin pre-treatments for reducing skin irritation resulting from friction against an area of skin, such as for example may be caused by the wearing of heels or other abrasive shoes.

The invention is not limited to specific fields-of-use. But, however, one example is the pre-treatment of skin for reducing risk of acquiring skin irritation resulting from friction against an area of skin as may be caused for example, and without limitation to the wearing of shoes, and will be described hereinafter with reference to this application.

### BACKGROUND OF THE INVENTION

The following discussion of the prior art is made without prejudice, and specifically does not constitute an admission that the invention has been previously known or used.

A common cause of skin irritation, resulting in skin chafing and/or blisters, includes friction. Known devices for treatment of skin against irritation resulting from friction or pressure are in the form of dressings (such as patches and/or bandages), which are typically applied after irritation has occurred. Such dressings can be adapted to slip over adjacent materials.

Naturally occurring, derivatized naturally-occurring and other polymers have been tested as liquid adhesive coatings for bandage applications and, in some cases, utilized commercially. Typical examples are nitrocellulose in various solvents (e.g., New Skin-Medtech Laboratories, Inc., Cody, Wyo.), aqar in water and diethylene glycol (U.S. Pat. No. 4,291,025) carrageenan and hydroxypropylmethyl cellulose in water (U.S. Pat. No. 4,318,746), and alginate in glycerin (U.S. Pat. No. 4,393,048). All of these natural polymers can support microbial growth, hence requiring the addition of a preservative or antimicrobial agent to the product. The liquid bandages based on water, diethylene glycol, glycerin, etc. are not only susceptible to microbial growth, but are often also slow drying due to high heats of vaporization; and are often water-sensitive, which can result in problems when used on areas of the body exposed to water. One commercial product, New Skin, does dry rapidly and is not water sensitive, but can cause stinging and further irritation of the skin upon application. None of these prior patents create a film designed to absorb extended periods of friction, and are all instead to serve as a wound covering post hoc irritation. Moreover, these bandage products are universally intended as “treatments” of wounds and not systems for “prevention” of chafing/blisters.

A few synthetic polymers have been patented for use as liquid adhesive coatings for bandage applications, most notably polymers containing 2-hydroxyethyl methacrylate (U.S. Pat. No. 4,303,066). These bandages based on the use of solvents can sting abraded areas; and the films can swell and wash off when in contact with water. For example, U.S. Pat. No. 4,569,784 claims an ointment, not a long-lasting bandage composed of an emulsion of water and silicone fluids, among other fluids. This reference can provide for an immediate soothing, but often not long-lasting treatment of the skin or mucous membranes. It also does not provide for fast drying, abrasion resistance, and other attributes which a polymer film can provide.

Additionally, traditional wound and surgical bandages, such as Band-Aids (Johnson & Johnson, New Brunswick, N.J.), comprised of film backings with adhesive, may contain silicones as part of either the adhesive or the backing (e.g. U.S. Pat. No. 4,650,817). These products are not applied as liquid adhesive coating where films form and adhere directly on the skin.

Liquid bandages for application to human and other skin have been described in the prior art for various purposes, including the delivery of medications to the skin surface. These include United States Patent Application 20060025516 entitled “Tissue protecting spray-on copolymeric film composition” to Shalby et al. for forming a film which can contain a bioactive agent including those to have antimicrobial, anesthetic, anti-inflammatory, and wound-healing activities. However, these products are not applied as liquid adhesive coatings where films form and adhere directly on the skin. This defect in the art may have led Salamone et al., (U.S. Pat. No. 4,987,893) to develop a liquid polymer-containing coating material which can act as a bandage or dressing to protect wounds post hac, and when applied in liquid form and air-dried on the already formed wound to form an adherent, solid protective film without significant stinging to the skin or mucous membranes of the user. The liquid polymer-containing coating material of U.S. Pat. No. 4,987,893 was noted to consist essentially of a siloxane-containing polymer and a solvent system comprising a polar solvent in small amount and a volatile liquid which is non-stinging to a user but provides bulk and formability to the liquid. Preferably, the polymer thereof is present from 1 to 40% by weight, the volatile liquid from 59.9 to 98.9% by weight and the polar solvent from 0.1 to 10% by weight. When the polar solvent is eliminated, the volatile liquid can be in amounts of 60 to 99%. The solvent is minimized to obtain flowability desired at the lowest solvent level feasible which purportedly minimizes stinging. The material forms a coating or bandage in the form of a dried film when applied to a surface or the skin of a user.

Moreover, U.S. Pat. No. 4,987,893 allegedly claims that combinations of alky siloxy siloxane-containing polymers admixed with liquid polydimethylsiloxanes are excellent non-stinging, non-irritating liquid coating materials for forming films which act as conformable bandages adhering to and protecting nails, skin, and mucous membrane existing wounds from yet further abrasion, contamination, and desiccation, while stopping pain from exposed nerve ends and allowing body fluid evaporation. However, to those skilled in the art of polymers, the compositions of U.S. Pat. No. 4,987,893 (1) are expected to result in low tear strength films because of being made of low-strength polysiloxane-based chains; (2) are not expected to form, in a timely manner, a solid film at the application site because of the low volatility of their major constituents; (3) are expected to yield hydrophobic films that have low or no tendency to adhere to moist tissue for sufficient periods of time—i.e., these films offer no advantage over regular bandages; and (4) are not expected to yield thin films on difficult-to-reach areas. Such undesirable features of U.S. Pat. No. 4,987,893 (and the prior art that preceded this patent) have led to the development of the amphiphilic, water-insoluble spray-on liquid compositions subject of this invention which provide low friction, flexible alternatives to regular and “liquid” bandages, that flex as the skin moves, strongly adhere to the skin, and stay intact as the barrier is continuously contacted by a shoe or item of clothing, for example.

Other examples of the application of film-forming compositions to the skin have been attempted for the purpose of reducing friction. One such example is United States Patent Application 2011/0250147 to Sher et al. entitled “Method and System for Reducing Skin Friction”, and the prior art cited therein. However, the Sher invention is generally not optimally effective—inasmuch as it relies solely upon the taught acrylic co-polymer supposedly but ineffectively to reduce friction and does not contain a specific active agent for the reduction of friction. Also, the Sher et al. chemical coating does not effectively flex with skin, and therefore tends to crack quickly upon application as the skin moved, rendering it ineffective. Moreover, the Sher et al. formulation is disqualified for human use as it contains substantial amounts of n-pentane, which is highly toxic.

See https://www.cdc.gov/niosh/idlh/109660.html

### Objects of the Invention

It is an object of the present invention to overcome or ameliorate at least one of the disadvantages of the prior art, and/or to provide useful alternatives.

It is a further object of the invention to provide a skin pre-treatment for reducing risk of acquiring chafing and/or a blister by providing a reduced coefficient of friction in a topically applied film-forming material.

It is a yet further object of the invention to provide compositions and related methods that:
1) Are intended as pre-treatment, not applied to wounds/irritation post hoc;
2) Are totally clear, so as not to be visible when worn in visible locations such as on the feet or ankles;
3) Are designed to be safe as a cosmetic (no known toxic solvents or aerosol agents);
4) Form a flexible coating that can move with skin by virtue of plasticizers that are compatible with, and pre-blended with the acrylic co-polymer;
5) Form a coating strong enough to endure continued friction/abrasion;
6) Utilize a friction-reducing agent;
7) Utilize an aerosolized formulation to enable rapid dry time; and
8) are initially water and sweat-resistant, adhere strongly enough to the skin to form a strong and stable coating, but yet will break down on its own naturally and/or will allow it to be washed off with water and washcloth using no chemical remover.

### SUMMARY OF THE INVENTION

According to an aspect of the invention there is provided a method of pre-treating an area of skin to reduce the risk of acquiring skin irritation, the method comprising the steps of:
providing an aerosol-based dispensing device containing a film-forming material; and
applying film-forming material to an area of skin, for pre-treating an area of skin to reduce friction and thereby reduce the risk of acquiring skin irritation that can result from friction against the area of skin.

Preferably, the method reduces the risk of acquiring skin chafing. More preferably, the method reduces the risk of acquiring a blister. According to an aspect of the invention there is provided a use of a film forming material for the preparation of a medicament for pre-treating an area of skin to reduce the risk of acquiring skin irritation that can result from friction against the area of skin. Preferably, the use reduces the risk of acquiring skin chafing. More preferably, the use reduces the risk of acquiring a blister.

Preferably, the film forming material is dissolved in a solvent. More preferably, a dispensing device containing the film forming material is pressurized such that the film forming material can be spray-applied in an aerosol format, and will dry in less than 90 seconds at average air temperatures and pressure.

Preferably, the film-forming material includes an acrylic co-polymer. More preferably, the film-forming material includes:
dimethyl ether at about 50 percent w/w;
acrylic co-polymer at about 4-10 percent w/w;
a plasticizer at about 1-5 percent w/w to enhance overall flexibility;
a friction-reducing agent at about 1-7 percent w/w;
ethyl, methyl, isobutyl or isopropyl or other acetates at about 35 percent w/w; and
with material avoidance of toxic organic solvents, such as the n-pentane solvent of the prior art (see https://www.cdc.gov/niosh/idlh/109660.html) Plasticizers can be selected from ethylene and propylene glycols and their esters, butyl cellosolve, butyl carbitol, Triethyl citrate, m,p-cresol propoxylate, alkyl benzyl phthalate, alkyl benzyl phthalate, dialkyl adipate, dioctyl phthalate, dibutyl phthalate or phosphate, diesters of phthalic and benzoic acids, triphenyl phosphate, trimethyl pentanyl diisobutyrate, acetyl tributyl citrate, ethyl tosylamide, and sucrose benzoate.

Acrylic co-polymers can be selected from Acrylates Copolymer, Ammonium Acrylates Copolymer, Ammonium/VA Acrylates Copolymer, Sodium Acrylates Copolymer, Ethylene/Acrylic Acid Copolymer, Ethylene/Calcium Acrylate Copolymer, Ethylene/Magnesium Acrylate Copolymer, Ethylene/Sodium Acrylate Copolymer, Ethylene/Zinc Acrylate Copolymer, Ethylene/Acrylic Acid/VA Copolymer, Acrylates/PVP Copolymer, Acrylates VA Copolymer, Steareth-10 Allyl Ether/Acrylates Copolymer, Acrylates/Steareth-50 Acrylate Copolymer, Acrylates/Steareth-20 Methacrylate Copolymer, Acrylates/Ammonium Methacrylate Copolymer, Styrene/Acrylates Copolymer, Styrene/Acrylates/Ammonium Methacrylate Copolymer, Ammonium Styrene/Acrylates Copolymer, Sodium Styrene/Acrylates Copolymer, Acrylates/Hydroxyesters Acrylates Copolymer, Methacryloyl Ethyl BEtaine/Acrylates, Lauryl Acrhylate/VA Copolymer, VA/Butyl Maleate/lsobomyl Acrylate Copolymer, Ethylene/Methacrylate Copolymer, Vinyl Caprolactam/PVP/Dimethylaminoethyl Methacrylate Copolymer, Sodium Acrylates/Acrolein Copolymer, PVP/Dimethylaminoethylmethacrylate Copolymer, AMP-Acrylates Copolymer, Polyacrylic Acid, Ammonium Polyacrylate, Potassium Aluminum Polyacrylate, Potassium Polyacrylate, Sodium Polyacrylate, urethane/Acrylate Copolymer.

The friction-reducing agent can be selected form a siloxane, can comprise a cyclosiloxane, the cyclosiloxane being selected from the group consisting of cyclomethicone, decamethylcyclopentasiloxane, cyclopentasiloxane, cyclotetrasiloxane, cyclohexasiloxane, and cycloheptasiloxane; a polyether siloxane copolymer; a Dimethicone Copolyol—i.e., polymers made from dimethicone and polyoxyethylene and/or polyoxypropylene, such as Dimethicone PEG-8 Adipate, Dimethicone PEG-8 Benzoate, Dimethicone PEG-7 Phosphate, Dimethicone PEG-10 Phosphate, Dimethicone PEG/PPG-20/23 Benzoate, Dimethicone PEG/PPG-7/4 Phosphate, Dimethicone PEG/PPG-12/4 Phosphate, PEG-3 Dimethicone, PEG-7 Dimethicone, PEG-8 Dimethicone, PEG-9 Dimethicone, PEG-10 Dimethicone, PEG-12 Dimethicone, PEG-14 Dimethicone, PEG-17 Dimethicone, PEG/PPG-3/10 Dimethicone, PEG/PPG-4/12 Dimethicone, PEG/PPG-6/11 Dimethicone, PEG/PPG-8/14 Dimethicone, PEG/PPG-14/4 Dimethicone, PEG/PPG-15/15 Dimethicone, PEG/PPG-16/2 Dimethicone, PEG/PPG-17/18 Dimethicone, PEG/PPG-18/18 Dimethicone, PEG/PPG-19/19 Dimethicone, PEG/PPG-20/6 Dimethicone, PEG/PPG-20/15 Dimethicone, PEG/PPG-20/20 Dimethicone, PEG/PPG-20/23 Dimethicone, PEG/PPG-20/29 Dimethicone, PEG/PPG-22/23 Dimethicone, PEG/PPG-22/24 Dimethicone, PEG/PPG-23/6 Dimethicone, PEG/PPG-25/25 Dimethicone and PEG/PPG-27/27 Dimethicone.

The acrylic copolymer can comprise one or more selected from the following: Acrylates Copolymer, Ammonium Acrylates Copolymer, Ammonium/VA Acrylates Copolymer, Sodium Acrylates Copolymer, Ethylene/Acrylic Acid Copolymer, Ethylene/Calcium Acrylate Copolymer, Ethylene/Magnesium Acrylate Copolymer, Ethylene/Sodium Acrylate Copolymer, Ethylene/Zinc Acrylate Copolymer, Ethylene/Acrylic Acid/VA Copolymer, Acrylates/PVP Copolymer, Acrylates VA Copolymer, Steareth-10 Allyl Ether/Acrylates Copolymer, Acrylates/Steareth-50 Acrylate Copolymer, Acrylates/Steareth-20 Methacrylate Copolymer, Acrylates/Ammonium Methacrylate Copolymer, Styrene/Acrylates Copolymer, Styrene/Acrylates/Ammonium Methacrylate Copolymer, Ammonium Styrene/Acrylates Copolymer, Sodium Styrene/Acrylates Copolymer, Acrylates/Hydroxyesters Acrylates Copolymer, Methacryloyl Ethyl BEtaine/Acrylates, Lauryl Acrhylate/VA Copolymer, VA/Butyl Maleate/lsobomyl Acrylate Copolymer, Ethylene/Methacrylate Copolymer, Vinyl Caprolactam/PVP/Dimethylaminoethyl Methacrylate Copolymer, Sodium Acrylates/Acrolein Copolymer, PVP/Dimethylaminoethylmethacrylate Copolymer, AMP-Acrylates Copolymer, Polyacrylic Acid, Ammonium Polyacrylate, Potassium Aluminum Polyacrylate, Potassium Polyacrylate, Sodium Polyacrylate, urethane/Acrylate Copolymer,

### BRIEF DESCRIPTION OF THE DRAWINGS

A preferred embodiment of the invention will now be described, by way of example only, with reference to the accompanying drawings in which:

FIG. 1 is a schematic view of a dispensing device containing a film forming material according to the invention as applied by way of example only and without limitation to the instep of a foot; and

FIG. 2 is a flowchart for a method of applying film forming material according to the invention.

### PREFERRED EMBODIMENTS OF THE INVENTION

According to an aspect of the invention there is provided a use of a film forming material for the preparation of a medicament for pre-treating an area of skin to reduce the risk of acquiring chafing and/or a blister, as herein described.

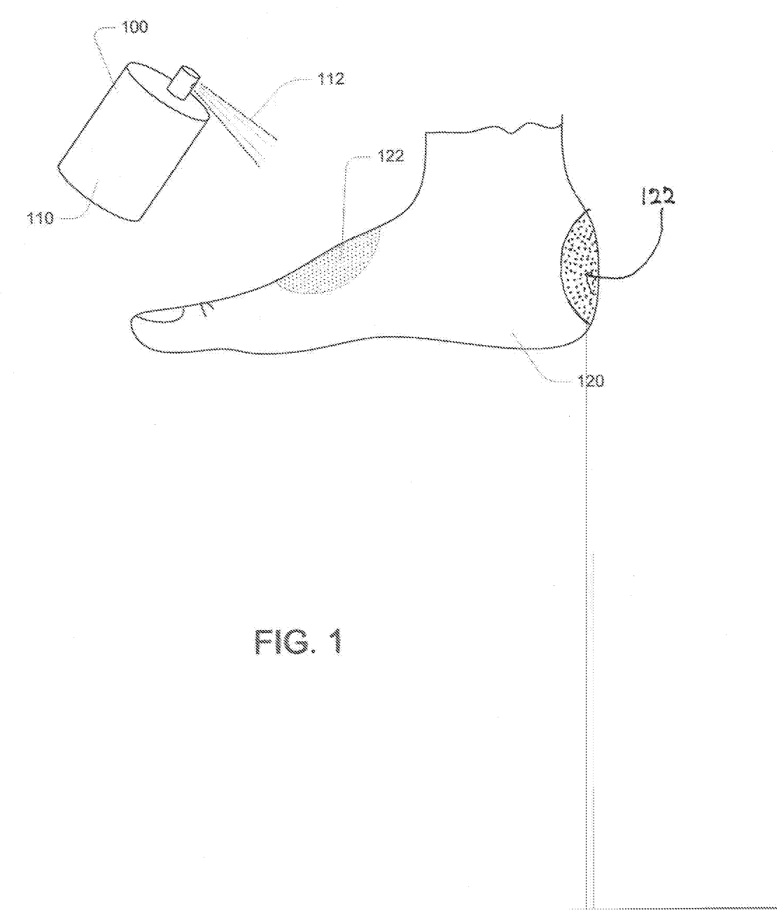

Referring to FIG. 1, a dispensing device 100 contains a film forming material 110. By way of example, the film forming material 110 is in the form of an acrylic co-polymer dissolved in non-toxic solvent, and pressurized for use in an aerosol form.

Using the aerosol dispensing device 100 to pre-treat an area of skin on a foot 120, a film forming material 110 can be sprayed 112 onto an area of skin 122 for forming a membrane. Although shown as applied to the instep therein, the compositions hereof may be applied to any area of the foot, particularly including the heel, or to any other part of the body. Preferably, the membrane is water resistant. More preferably, the membrane is air permeable. Wherefore, it will be appreciated that the aerosol dispensing device 100 can pre-treat almost any area of the body to reduce the risk of acquiring chafing and/or a blister.

It will be appreciated that, by way of example only, pre-treatment can cause the formation of a water-resistant air-permeable membrane over an area of skin to provide friction protection and thereby reduce the risk of acquiring chafing and/or a blister.

In an embodiment, the film forming material is in the form of an acrylic co-polymer dissolved in a non-toxic solvent and pressurized in an aerosol format. It will be appreciated that other film-forming materials can be used in accordance with the teaching herein, for pre-treating an area of skin to reduce friction and thereby reduce the risk of acquiring chafing and/or a blister.

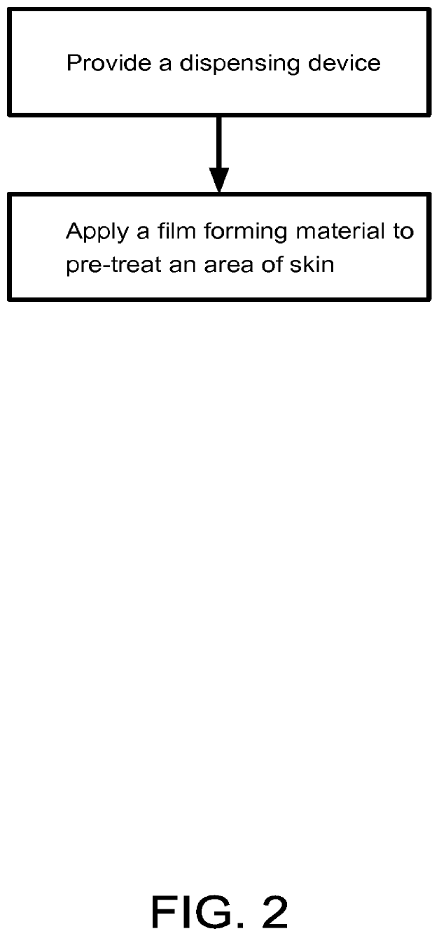

Referring to FIG. 2, an example method 200 of applying a film forming material, may include:
STEP 210: providing a dispensing device containing a film-forming material; and
STEP 220: applying a film-forming material to an area of skin, for pre-treating an area of skin to reduce friction and thereby reduce the risk of acquiring chafing and/or a blister.

In one preferred embodiment, applying a film forming material comprises spraying a non-irritating solvent-based film forming material onto an area of intact skin for reducing skin friction, which is a common cause of chafing and blisters. It will be appreciated that the method 200 can be both effective and convenient in reducing skin friction.

It will be appreciated that the illustrated method provides a skin pre-treatment for reducing risk of acquiring skin irritation that can result from friction against the area of skin. Preferably, the method reduces the risk of acquiring skin chafing. More preferably, the method reduces the risk of acquiring a blister.

Although the invention has been described with reference to specific examples, it will be appreciated by those skilled in the art that the invention may be embodied in many other forms.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Any one of the terms comprising, comprised of or which comprises is an open term that means including at least the elements/features that follow, but not excluding others. Thus, the term comprising should not be interpreted as being limitative to the means or elements or steps listed thereafter. For example, the scope of the expression “a device comprising A and B” should not be limited to devices consisting only of elements A and B. Any one of the terms including or which includes or that includes as used herein is also an open term that also means including at least the elements/features that follow the term, but not excluding others. Thus, “including” is synonymous with and means “comprising”.

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

Similarly, it should be appreciated that in the above description of exemplary, preferred or alternative embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, inventive aspects lie in less than all features of a single foregoing disclosed embodiment. Thus, the claims subsequently hereof are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, any of the claimed embodiments can be used in any combination.

In the description provided herein, numerous specific details are set forth. However, it is understood that embodiments of the invention may be practiced without these specific details. In other instances, well-known methods, structures and techniques have not been shown in detail in order not to obscure an understanding of this description.

Thus, while there has been described what are believed to be preferred embodiments of the invention, those skilled in the art will recognize that other and further modifications may be made thereto without departing from the spirit of the invention, and it is intended to claim all such changes and modifications as fall within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

What is claimed is:

1. A method of reducing frictional engagement upon the skin of a human being, comprising:
providing a liquid comprising an effective amount of a non-toxic water-insoluble occlusive film-forming polymer in a topically-acceptable, non-toxic medium, a friction-reducing agent, and a carrier solvent;
applying the liquid to the skin of a human being; and
permitting the carrier liquid to dry, and thereby to form a friction reducing film.

2. The method of claim 1, wherein the liquid is applied to the skin of the human foot.

3. The method of claim 1, wherein the liquid contains at least one modifier.

4. The method of claim 1, wherein the liquid contains at least one defoamer.

5. The method of claim 1, wherein the liquid contains at least one plasticizer selected from the group consisting of ethylene and propylene glycols and their esters, butyl cellosolve, butyl carbitol, Triethyl citrate, m,p-cresol propoxylate, alkyl benzyl phthalate, alkyl benzyl phthalate, dialkyl adipate, dioctyl phthalate, dibutyl phthalate or phosphate, diesters of phthalic and benzoic acids, triphenyl phosphate, trimethyl pentanyl diisobutyrate, acetyl tributyl citrate, ethyl tosylamide, and sucrose benzoate.

6. The method of claim 5, wherein the plasticizer is present at about 1-5 percent w/w.

7. The method of claim 1, wherein the liquid contains includes an aerosol propellant.

8. The method of claim 1, wherein the aerosol propellant is dimethyl ether.

9. The method of claim 7, wherein the aerosol propellant comprises dimethyl ether and is present at about 50 percent w/w; the polymer comprises acrylic co-polymer and is present at about 4-10 percent w/w; the friction-reducing agent is present at about 1-7 percent w/w; and the carrier solvent is present at about 35 percent w/w.

10. The method of claim 1, wherein the carrier solvent is selected from the group consisting of ethyl acetate, methyl acetate, isobutyl acetate, and isopropyl acetate.

11. The method of claim 1, with material avoidance of toxic organic solvents.

12. The method of claim 1, wherein the Acrylic co-polymers is selected from the group consisting of Acrylates Copolymer, Ammonium Acrylates Copolymer, Ammonium/VA Acrylates Copolymer, Sodium Acrylates Copolymer, Ethylene/Acrylic Acid Copolymer, Ethylene/Calcium Acrylate Copolymer, Ethylene/Magnesium Acrylate Copolymer, Ethylene/Sodium Acrylate Copolymer, Ethylene/Zinc Acrylate Copolymer, Ethylene/Acrylic Acid/VA Copolymer, Acrylates/PVP Copolymer, Acrylates VA Copolymer, Steareth-10 Allyl Ether/Acrylates Copolymer, Acrylates/Steareth-50 Acrylate Copolymer, Acrylates/Steareth-20 Methacrylate Copolymer, Acrylates/Ammonium Methacrylate Copolymer, Styrene/Acrylates Copolymer, Styrene/Acrylates/Ammonium Methacrylate Copolymer, Ammonium Styrene/Acrylates Copolymer, Sodium Styrene/Acrylates Copolymer, Acrylates/Hydroxyesters Acrylates Copolymer, Methacryloyl Ethyl BEtaine/Acrylates, Lauryl Acrhylate/VA Copolymer, VA/Butyl Maleate/lsobomyl Acrylate Copolymer, Ethylene/Methacrylate Copolymer, Vinyl Caprolactam/PVP/Dimethylaminoethyl Methacrylate Copolymer, Sodium Acrylates/Acrolein Copolymer, PVP/Dimethylaminoethylmethacrylate Copolymer, AMP-Acrylates Copolymer, Polyacrylic Acid, Ammonium Polyacrylate, Potassium Aluminum Polyacrylate, Potassium Polyacrylate, Sodium Polyacrylate, urethane/Acrylate Copolymer.

13. The method of claim 1, wherein the friction-reducing agent is a siloxane selected from the group consisting of:
a cyclosiloxane selected from the group consisting of cyclomethicone, decamethylcyclopentasiloxane, cyclopentasiloxane, cyclotetrasiloxane, cyclohexasiloxane, and cycloheptasiloxane;
a polyether siloxane copolymer; and
a Dimethicone Copolyol polymer, selected from the group consisting of Dimethicone PEG-8 Adipate, Dimethicone PEG-8 Benzoate, Dimethicone PEG-7 Phosphate, Dimethicone PEG-10 Phosphate, Dimethicone PEG/PPG-20/23 Benzoate, Dimethicone PEG/PPG-7/4 Phosphate, Dimethicone PEG/PPG-12/4 Phosphate, PEG-3 Dimethicone, PEG-7 Dimethicone, PEG-8 Dimethicone, PEG-9 Dimethicone, PEG-10 Dimethicone, PEG-12 Dimethicone, PEG-14 Dimethicone, PEG-17 Dimethicone, PEG/PPG-3/10 Dimethicone, PEG/PPG-4/12 Dimethicone, PEG/PPG-6/11 Dimethicone, PEG/PPG-8/14 Dimethicone, PEG/PPG-14/4 Dimethicone, PEG/PPG-15/15 Dimethicone, PEG/PPG-16/2 Dimethicone, PEG/PPG-17/18 Dimethicone, PEG/PPG-18/18 Dimethicone, PEG/PPG-19/19 Dimethicone, PEG/PPG-20/6 Dimethicone, PEG/PPG-20/15 Dimethicone, PEG/PPG-20/20 Dimethicone, PEG/PPG-20/23 Dimethicone, PEG/PPG-20/29 Dimethicone, PEG/PPG-22/23 Dimethicone, PEG/PPG-22/24 Dimethicone, PEG/PPG-23/6 Dimethicone, PEG/PPG-25/25 Dimethicone and PEG/PPG-27/27 Dimethicone.
